(19) United States
(12) Patent Application Publication
(10) Pub. No.: US 2007/0071651 A1
(43) Pub. Date: Mar. 29, 2007

(54) DETECTING DEVICE FOR HYDROGEN HALIDE GAS AND ABSORBING APPARATUS FOR HYDROGEN HALIDE GAS

(76) Inventors: Yasuhiro KATO, Yokohama-shi (JP); Masahiro Kato, Naka-gun (JP); Kenji Essaki, Kawasaki-shi (JP); Toshihiro Imada, Yokohama-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 11/534,801

(22) Filed: Sep. 25, 2006

(30) Foreign Application Priority Data
Sep. 29, 2005 (JP) 2005-284061

(51) Int. Cl. G01N 33/00

(52) U.S. Cl. 422/83

(57) ABSTRACT

A detecting device for a hydrogen halide gas, includes an insulating support, a detecting member supported on the insulating support and containing an absorbent which reacts with the hydrogen halide gas to produce water, and a pair of electrodes attached respectively to both ends of the detecting member and configured to measure a change in an electric resistance value or an electrostatic capacitance of the detecting member, caused by the production of water due to a reaction between the hydrogen halide gas and the absorbent in the detecting member.

### CROSS-REFERENCE TO RELATED APPLICATIONS

This application is based upon and claims the benefit of priority from prior Japanese Patent Application No. 2005-284061, filed Sep. 29, 2005, the entire contents of which are incorporated herein by reference.

### BACKGROUND OF THE INVENTION

1. Field of the Invention

The present invention relates to a detecting device for a hydrogen halide gas and an absorbing apparatus for a hydrogen halide gas.

2. Description of the Related Art

For example, the manufacture of semiconductor devices includes processes in which various types of films are subjected to dry etching. In these processes, various types of dry etching gases for respective films are used. For example, a hydrogen halide gas such as hydrogen fluoride gas is used solely or in mixture with some other etching gas or inert gas.

Hydrogen halide gases are highly toxic and dangerous, and therefore it is important to detect leakage of gas from a pipe or the like, thereby making it possible to prevent the deterioration of work environment.

Conventionally, such a hydrogen halide gas is eliminated by the following method. That is, a absorbent made of granules containing alkali components is filled into a reaction column, and a gas to be treated, which contains a hydrogen halide gas is circulated in the reaction column to react the absorbent and the hydrogen halide gas with each other, thereby eliminating the hydrogen halide gas. However, when reacted with a certain amount of hydrogen halide gas, the absorbent does not further react, that is, it reaches the so-called breakthrough. In this case, hydrogen halide gas flows out from the outlet of the reaction column to the environment, thereby possibly damaging the surrounding environment. In order to avoid this, it is necessary to measure the concentration of the hydrogen halide gas in the gas discharged from the outlet of the reaction column to accurately detect the breakthrough of the absorbent for the hydrogen halide gas.

For the above-described situation, it is conventionally known that a hydrogen halide gas can be detected by using a constant-potential electrolysis type gas sensor or a detecting tube method. Jpn. Pat. Appln. KOKAI Publication No. 2004-333164 discloses a small-sized and easily-assembled constant-potential electrolysis type gas sensor.

However, the constant-potential electrolysis type gas sensor entails such a drawback that it requires an electrolytic solution, thereby making the structure complicated. On the other hand, the detecting tube method entails such a drawback that it requires to sample the gas from the atmosphere of the hydrogen halide gas for each detecting operation, thereby making the detecting operation troublesome.

### BRIEF SUMMARY OF THE INVENTION

According to the first aspect of the present invention, there is provided a detecting device for a hydrogen halide gas, comprising:

an insulating support;

a detecting member supported on the insulating support and containing an absorbent which reacts with the hydrogen halide gas to produce water; and

a pair of electrodes attached respectively to both ends of the detecting member and configured to measure a change in an electric resistance value or an electrostatic capacitance of the detecting member, caused by the production of water due to a reaction between the hydrogen halide gas and the absorbent in the detecting member.

According to the second aspect of the present invention, there is provided an absorbing apparatus for a hydrogen halide gas, comprising:

a cylindrical absorbing column made of an insulating material, through which a gas to be treated containing a hydrogen halide gas is allowed to flow, and having an inlet and an outlet of the gas to be treated;

a plurality of absorbents filled in the absorbing column and which reacts with the hydrogen halide gas to produce water; and

a pair of electrodes provided at a section of the absorbent situated on at least the outlet side of the absorbing column, and configured to measure a change in an electric resistance value or an electrostatic capacitance of the absorbents, caused by the production of water due to a reaction between the hydrogen halide gas and the absorbents.

### BRIEF DESCRIPTION OF THE SEVERAL VIEWS OF THE DRAWING

FIG. 1 is a diagram showing a perspective view of a detecting device for hydrogen halide gas, according to the first embodiment of the present invention;

FIG. 2 is a diagram showing a perspective view of the detecting device for hydrogen halide gas, shown in FIG. 1, when absorbent in the detecting member of the device reacts with hydrogen chloride gas;

FIG. 3 is a diagram showing a perspective view of an alternative version of the detecting device for hydrogen halide gas;

FIG. 4 is a diagram showing a cross sectional view of an absorbing apparatus for hydrogen halide gas, according to the second embodiment of the present invention;

FIG. 5 is a diagram showing the absorbing apparatus for hydrogen halide gas, shown in FIG. 4, when the absorbents located near a pair of electrodes of the apparatus reacts with hydrogen chloride gas;

FIG. 6 is a diagram showing the change in an electric resistance value between the electrodes along with the detection time in Example 1; and

FIG. 7 is a diagram showing the change in an electric resistance value between the electrodes along with the time in which the hydrogen halide gas is allowed to flow through the cylindrical absorbing column, in Example 2.

### DETAILED DESCRIPTION OF THE INVENTION

Embodiments of the present invention will now be described with reference to accompanying drawings.

(First Embodiment)

The detection device for hydrogen halide gas, according to the first embodiment comprises an insulating support. A detecting member is supported on the insulating support. The detecting member is contained an absorbent that creates water when reacting with a hydrogen halide gas. A pair of electrodes are attached respectively to both ends of the detecting member and are configured to measure a change in an electric resistance value or an electrostatic capacitance of the absorbing member, caused by the creation of water due to the reaction of the hydrogen halide gas by the absorbent in the absorbing member.

The insulating support may be a plate made of a general purpose plastic such as polyethylene or polypropylene, or a ceramic plate made of, for example, alumina.

The absorbent contains at least one selected from the group consisting of lithium composite oxides and hydroxides of alkaline earth metals. The absorbent may further contain a binder resin in addition to these compounds. Usable examples of the binder resin are polyvinyl alcohol (PVA), polyvinyl butyral (PVB), wax, paraffin and carboxymethylcellulose (CMC). It is preferable that the binder resin should be contained in the absorbent at a ratio of 0.1 to 20% by weight.

Examples of the lithium composite oxides are lithium silicate, lithium zirconate, lithium ferrite, lithium nickelate, lithium titanate and lithium aluminate, each of which can be used solely or in the form of a mixture of these.

Examples of the hydroxides of alkaline earth metals are magnesium hydroxide, calcium hydroxide, strontium hydroxide and barium hydroxide, each of which can be used solely or in the form of a mixture of these.

When, for example, a hydrogen chloride gas is used as the hydrogen halide gas, each of the lithium composite oxides and the hydroxides of alkaline earth metals reacts with the hydrogen chloride gas as presented in the formulas (1) to (11) below to be absorbed.
Li₄SiO₄(s)+4HCl→2LiCl(s)+SiO₂(s)+2H₂O (1)
Li₂SiO₃(s)+2HCl→2LiCl(s)+SiO₂(s)+H₂O (2)
Li₂ZrO₃(s)+2HCl→2LiCl(s)+ZrO₂(s)+H₂O (3)
2LiFeO₂(s)+2HCl→2LiCl(s)+Fe₂O₃(s)+H₂O (4)
2LiNiO₂(s)+2HCl→2LiCl(s)+Ni₂O₃(s)+H₂O (5)
Li₂TiO₃(s)+2HCl→2LiCl(s)+TiO₂(s)+H₂O (6)
2LiAlO₂(s)+2HCl→2LiCl(s)+Al₂ZO₃(s)+H₂O (7)
Mg(OH)₂(s)+2HCl→MgCl₂(s)+2H₂O (8)
Ca(OH)₂(s)+2HCl→CaCl₂(s)+2H₂O (9)
Sr(OH)₂(s)+2HCl→SrCl₂(s)+2H₂O (10)
Ba(OH)₂(s)+2HCl→BaCl₂(s)+2H₂O (11)

As presented by the above formulas, the lithium composite oxides and the hydroxides of alkaline earth metal can react with the hydrogen chloride gas to be absorbed. At the same time, the reaction generates water to make the absorbent into a mud-like state.

It should be noted that there are two types of lithium silicate as indicated by the formulas (1) and (2). Theoretically, the lithium silicate (Li₄SiO₄) expressed in the formula (1) is capable of absorbing the hydrogen chloride gas twice as much (in molar ratio) as compared to the lithium composite oxides indicated in the formulas (2) to (7). Thus, the lithium silicate (Li₄SiO₄) is appropriate to absorb hydrogen halide gas such as hydrogen chloride gas.

It is preferable that the detecting member is formed to have a structure in which a plurality of absorbents made of granules are supported on the insulating support such as to be in contact with the pair of electrodes. The granules are of a spherical shape, a three-dimensional body close to sphere, an ellipsoid, a cylinder or a prism such as a square pillar. It is preferable that the average diameter or thickness should be 1 μm to 3 mm, since with such size, a large contact area with the hydrogen halide gas can be obtained, making it possible to achieve a quick detection of the hydrogen halide gas.

The electrodes are made of a metal such as Cu, Ni or Au.

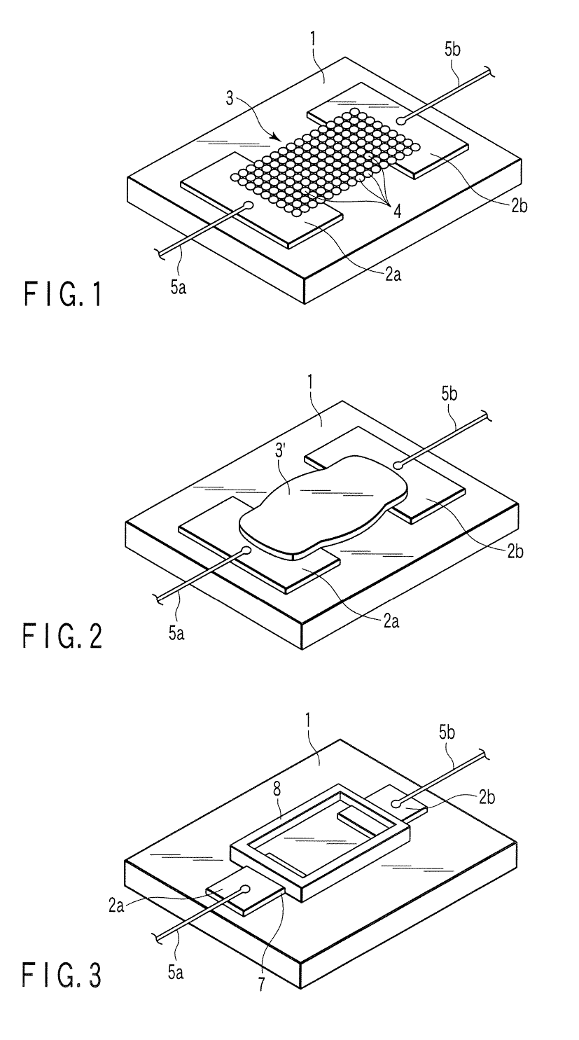

Next, the detecting device for hydrogen halide gas according to the first embodiment will now be described in detail with reference to FIG. 1.

A pair of electrodes 2a and 2b are formed on a plate-like insulating support 1 such as a predetermined distance apart from each other. A ribbon detecting member 3 is formed by spreading a number of granular absorbents 4 (for example, granular lithium silicate: Li₄SiO₄) on the insulating support 1 such as to fall on the pair of electrodes 2a and 2b. These granular absorbents react with hydrogen halide gas to produce water. Leads 5a and 5b are connected respectively to the pair of electrodes 2a and 2b each by one end, and the other ends are connected to a resistance meter or electrostatic capacitance meter, either one of which is not shown in the figure.

A method of detecting a hydrogen halide gas (such as hydrogen chloride gas) by using a detecting device for hydrogen halide gas, shown in FIG. 1, will now be described.

The insulating support 1 is placed in a place where hydrogen chloride gas to be measured. When a gas to be measured, which contains hydrogen chloride, flows and passes on the insulating support 1, the granular absorbents, for example, lithium silicate (Li₄SiO₄) granules, which form the ribbon detecting member 3, are brought into contact with the hydrogen chloride gas. On contact, the hydrogen chloride gas quickly react with the lithium silicate granules as presented in the formula (1) to be absorbed therein, and then water is produced as a result of the reaction. Due to the creation of water, the granular absorbents transform into the detecting member 3′ of muddy absorbents as shown in FIG. 2, and thus, for example, the resistance value changes. More specifically, the muddy detecting member 3′ has a resistance value lower as compared to that of the ribbon detecting member 3 of the granular lithium silicate before reacted with the hydrogen chloride gas (before the absorption of water). Based on this mechanism, the change in resistance value between the electrodes 2a and 2b is monitored using the resistant meter (not shown) connected via the leads 5a and 5b to the pair of electrodes 2a and 2b contacting the vicinities of both ends of the muddy detecting member 3′, to detect the hydrogen chloride gas flowing into the atmosphere of the place to be measured.

The hydrogen halide gas to be detected is not limited to hydrogen chloride, but it may be hydrogen fluoride, hydrogen bromide, hydrogen iodide, or the like.

As described above, according to the first embodiment, the hydrogen chloride gas flowing into the atmosphere of the place to be measured, reacts with the detecting member contained the gas absorbents, and further the reaction produces water to make the absorbents muddy. Due to the transformation of the material into the muddy state, the resistance value (or electrostatic capacitance) between the pair of electrodes changes. By monitoring the change in the resistance, it is possible to accurately detect hydrogen halide gas leaking from a place to be measured, such as a pipe, without requiring a complicated operation such as sampling. Thus, a hydrogen halide gas detecting device with a simple structure can be provided.

It should be noted that the hydrogen halide gas detecting device according to the first embodiment may take a structure as shown in FIG. 3. That is, a pair of electrodes 2a and 2b are fixed to be a predetermined distance apart from each other onto the insulating support 1. A rectangular frame body 8 made of an insulating material such as plastics and having notches 7 in lower sections of respective opposing side walls of the frame body is fixed onto the insulating support 1 such that the notches 7 engage respectively with the pair of electrodes 2a and 2b at their central portions. A number of granular absorbents (for example, granular lithium silicate: Li₄SiO₄) are filled into the frame body 8 to form a ribbon detecting member (not shown). With such a structure as shown in FIG. 3, a number of granular absorbents are filled in the frame body 8 fixed onto the insulating support 1, and thus a ribbon detecting member, which is not shown in the figure, is formed within the frame body 8. Therefore, the ribbon detecting member can be surely connected to the pair of electrodes 2a and 2b each by a constant area at the same time. Consequently, a hydrogen halide gas detecting device of an even higher handleability can be realized.

(Second Embodiment)

The detection device for hydrogen halide gas, according to the second embodiment comprises a cylindrical absorbing column made of an insulating material. A gas to be treated containing a hydrogen halide gas is allowed to pass through the absorbing column. The absorbing column has an inlet and an outlet of the gas to be treated. A plurality of absorbent are filled into the absorbing column and are producing water when reacted with hydrogen halide gas. A pair of electrodes is provided in a section of the absorbents situated on at least on an outlet side of the absorbing column, and are configured to measure a change in an electric resistance value or an electrostatic capacitance of the absorbents, caused by the creation of water due to the reaction of the hydrogen halide gas by the absorbents at the section.

The cylindrical absorbing column may be made of a general purpose plastic such as polyethylene or polypropylene, or a ceramic plate made of, for example, alumina.

The absorbents each contain at least one selected from the group consisting of lithium composite oxides and hydroxides of alkaline earth metals. The absorbent may further contain a binder resin in addition to these compounds. Usable examples of the binder resin are polyvinyl alcohol (PVA), polyvinyl butyral (PVB), wax, paraffin and calboxymethylcellulose (CMC). It is preferable that the binder resin should be contained in the absorbent at a ratio of 0.1 to 20% by weight.

Examples of the lithium complex oxide and the hydroxides of alkaline earth metals are similar to those described in connection with the first embodiment. Further, each of the lithium composite oxides and the hydroxides of alkaline earth metals reacts with, for example, a hydrogen chloride gas as presented in the formulas (1) to (11) set forth above to be absorbed. Of these examples, the lithium silicate (Li₄SiO₄) is preferable since it is capable of absorbing the hydrogen chloride gas more as compared to the other lithium composite oxides indicated.

It is preferable that the absorbents should be filled in the absorbing column in the granular form of a spherical shape, a three-dimensional body close to sphere, an ellipsoid, a cylinder or a prism such as a square pillar. It is also preferable that the average diameter or thickness should be 50 μm to 30 mm, since with such a size, a large contact area with the hydrogen halide gas can be obtained. Further, with such a size, it is possible to reduce the pressure loss of the to-be-treated gas flowing between the granular absorbents. Consequently, the hydrogen halide gas can be absorbed and removed at a high efficiency.

The electrodes are made of a metal such as Cu, Ni or Au.

The pair of electrodes is provided at the section of the absorbents located on the to-be-processed gas outlet side of the absorbing column. It is alternatively possible that two or more pairs of electrodes are provided from the outlet side towards the inlet side of the absorbing column. It is desirable that a pair of electrodes is provided at a section of the absorbents located in a range of 1/10 to ½ of the filling height of the absorbents from the outlet of the absorbing column.

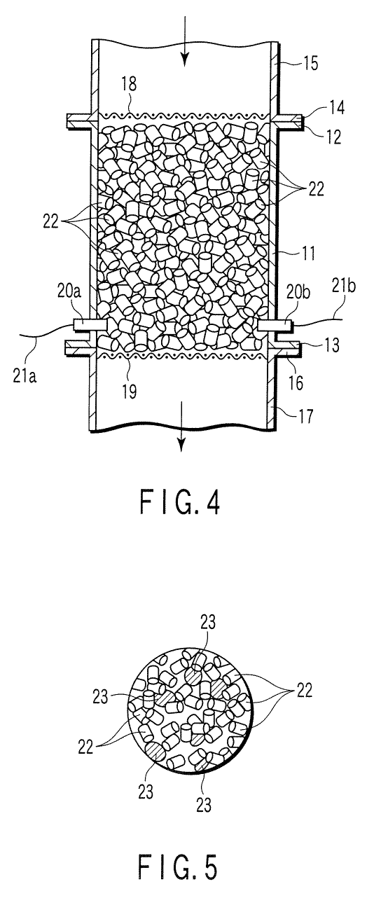

Next, the absorbing apparatus for hydrogen halide gas according to the second embodiment will now be described in detail with reference to FIG. 4.

A cylindrical absorbing column 11 has flanges 12 and 13 at its upper and lower sections. Upper and lower sections of the absorbing column 11 are respectively formed an inert and an outlet. An inlet-side pipe 15 having a flange 14 at its lower end is coupled to the flange 12 in the upper section of the cylindrical absorbing column 11 via the lower-end flange 14. An outlet-side pipe 17 having a flange 16 at its upper end is coupled to the flange 13 in the lower section of the cylindrical absorbing column 11 via the upper-end flange 14. Round mesh plates 18 and 19 are provided respectively to the inner surface at the lower end of the inlet-side pipe 15 and the inner surface at the upper end of the outlet-side pipe 17. It should be noted that the cylindrical absorbing column having the flanges 12 and 13 at its upper and lower sections is made of an insulating material such as plastic or ceramic.

A pair of slender electrodes 20a and 20b are inserted to sections of the absorbing column 11 located near the outlet such as to oppose each other. Leads 21a and 21b are connected respectively to the pair of electrodes 20a and 20b each by one end, and the other ends are connected to a resistance meter or electrostatic capacitance meter, either one of which is not shown in the figure. A number of granular absorbent 22, for example, lithium silicate (Li₄SiO₄) granules, which react with hydrogen halide gas to produce water, are filled into the absorbing column 11 to such a height that is sufficient to bury the electrodes 20a and 20b.

A method of absorbing and removing a hydrogen halide gas (such as hydrogen chloride gas) by using an absorbing device for hydrogen halide gas, shown in FIG. 4, will now be described.

A gas to be treated, which contains a hydrogen chloride gas is supplied via the inlet-side pipe 15 to an inlet of the cylindrical absorbing column 11 filled with a number of lithium silicate granules 22 and passed therein. During this operation, the hydrogen chloride gas in the to-be-treated gas reacts with the lithium silicate granules 22 as presented in the formula (1) presented before to be absorbed as solid lithium chloride therein, and then water is produced as a result of the reaction in the inlet-side section of the absorbing column 11 in which the lithium silicate granules 22 are filled. As the supply and flow of the to-be-treated gas to the absorbing column 11 is continued, the reaction site between the hydrogen chloride gas in the treated gas and the lithium silicate granules 22 shifts from the inlet-side of the absorbing column 11 to the outlet side.

Further, as the flow of the to-be-treated gas to the absorbing column 11 is continued and the reaction site between the hydrogen chloride gas and the lithium silicate granules 22 reaches a section near the outlet of the absorbing column 11 where the pair of slender electrodes 20a and 20b are located, the lithium silicate granules 22 themselves narrow down due to the reaction presented in the formula (1) in the section filled with the lithium silicate granules 22 as shown in FIG. 5, and further a muddy material 23 is created around the lithium silicate granules due to water created by the reaction. As a result, for example, an electric resistance value between the pair of electrodes 20a and 20b changes. More specifically, the resistance value between the electrodes 20a and 20b located in the section formed muddy material 23 lowers as compared to that between the pair of electrodes 20a and 20b before reacting with the hydrogen chloride gas (that is, before absorbed). Based on this mechanism, the change in an electric resistance value between the electrodes 20a and 20b is monitored using the resistant meter (not shown) connected via the leads 21a and 21b to the pair of electrodes 20a and 20b. Thus, it is possible to detect that the reaction no longer proceeds in the section filled with the lithium silicate granules 22 and where the pair of electrodes 20a and 20b are located, that is, the reaction has reached the so-called breakthrough. Immediately after detecting the breakthrough, the supply of the treated gas containing the hydrogen chloride gas is stopped.

It should be noted that examples of the to-be-treated gas are a hydrogen chloride gas discarded as waste gas resulting after cleaning process, and a cleaning gas containing an insert gas such as nitrogen. The hydrogen halide gas in the to-be-treated gas is not limited to hydrogen chloride, but it may be hydrogen fluoride, hydrogen bromide, hydrogen iodide, or the like.

As described above, according to the second embodiment, the breakthrough that occurs during the elimination of a hydrogen halide gas with absorbents by reaction can be accurately detected. With such an operation, it is possible to provide an absorbing apparatus for hydrogen halide gas that can prevent an unreacted hydrogen halide gas from being discharged from an outlet of the reaction column.

Examples of the present invention will now be described with reference to the above-mentioned drawings.

### EXAMPLE 1

A silicon oxide powder having an average grain diameter of 1 μm and a lithium carbonate powder having an average grain diameter of 1 μm were mixed together at a molar ratio of 1:2 to prepare a powder mixture. The powder mixture was baked at a temperature of 900° C. in the atmosphere and thus a plurality of granular absorbents made of lithium silicate (Li₄SiO₄) granules having an average grain diameter of 1 μm was obtained.

On the other hand, gold paste was applied on an plate-like insulating support 1 made of alumina and then dried, thereby forming a pair of electrodes 2a and 2b with a distance of 25 mm therebetween. The granular absorbent 4 thus obtained was spread on the plate-like insulating support 1 in amount of 0.5 g such as to fall on the pair of electrodes 2a and 2b, thereby forming a ribbon detecting member 3. Leads 5a and 5b are connected respectively to the pair of electrodes 2a and 2b by one end, and the other ends of the leads 5a and 5b were connected to a resistance meter, which is not shown in the figure. Thus, a detecting device for hydrogen halide gas shown in FIG. 1 was manufactured.

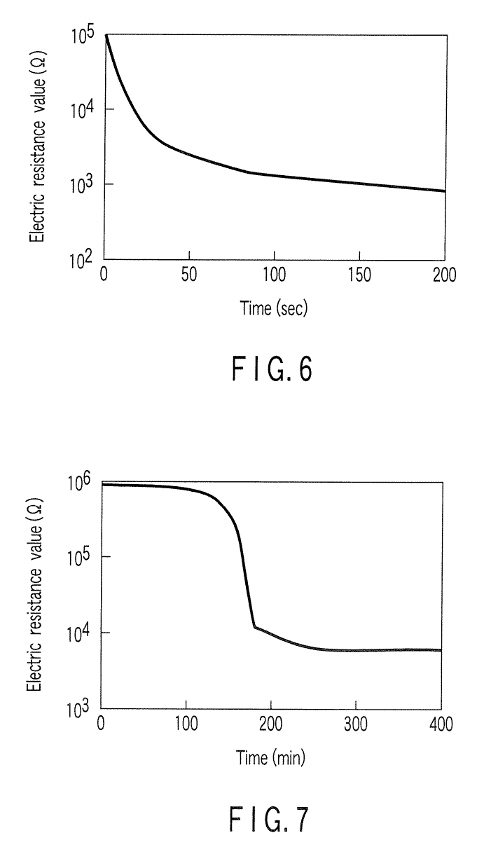

The obtained detecting device of Example 1 was placed in a cylinder having a diameter of 10 cm, and a mixture gas of 99% of nitrogen gas and 1% of HCl gas was allowed flow through the cylinder at 100 mL/min. During this operation, an electric resistance value between the electrodes 2a and 2b was measured continuously with the resistance meter (not shown). The change in electric resistance value along with time is plotted in FIG. 6.

As is clear from FIG. 6, it is confirmed that the detecting device of Example 1 can detect that the gas flowing through the cylinder contains hydrogen chloride gas since the resistance value lowers as the hydrogen chloride gas comes in contact with the detecting member 3.

### Example 2

Lithium silicate granules obtained in Example 1 and PVA as a binder resin were mixed together at a weight ratio of 1:0.01 to prepare a mixture. The mixture was rotated in the presence of water by the rotation method, thereby obtaining absorbents having shapes very close to spheres and having an average grain diameter of 500 μm.

As shown in FIG. 4, a cylindrical absorbing column 11 made of alumina and having a diameter of 25 mm and a height of 100 mm, with flanges 12 and 13 at its upper and lower sections was prepared. An outlet-side pipe 17 having a round mesh plate 19 at the inner surface of its upper end was coupled to the flange 13 in the lower section of the cylindrical absorbing column 11 via the upper-end flange 16. The cylindrical absorbing column 11 was then filled with an amount of 10 g of the absorbents 22. Subsequently, an inlet-side pipe 17 having a round mesh plate 18 at the inner surface of its lower end was coupled to the flange 12 in the upper section of the cylindrical absorbing column 11 via the lower-end flange 14. A pair of slender electrodes 20a and 20b are inserted to sections of the cylindrical absorbing column 11 at a section 10 mm above the lower-end flange 13 such as to oppose each other. Leads 21a and 21b are connected respectively to the pair of electrodes 20a and 20b each by one end, and the other ends are connected to a resistance meter, which is not shown in the figure. Thus, an absorbing apparatus for hydrogen halide gas was assembled.

A mixture gas of 98% of nitrogen gas and 2% of HCl gas was allowed flow through the cylindrical absorbing column 11 obtained in Example 2 via the inlet-side pipe 15 at a rate of 100 mL/min. During this operation, the electric resistance value between the pair of electrodes 20a and 20b was measured continuously with the resistance meter (not shown). The change in electric resistance value along with time is plotted in FIG. 7.

As is clear from FIG. 7, in an initial stage of the flow of the mixture gas containing hydrogen chloride gas, the reaction between the absorbents 22 and HCl proceeded in the vicinity of the inlet side of the cylindrical absorbing column 11, and the resistance value of the absorbent located near the pair of electrodes 20a and 20b, which are provided on the outlet side of the cylindrical absorbing column 11, did not change. The flow of the mixture gas was continued and after 160 minutes, the resistance value lowered. That is, the reaction between HCl and the absorbent 22 near the pair of electrodes 20a and 20b on the outlet side of the cylindrical absorbing column 11 proceeded, and it was detected that the absorbent 22 reached the breakthrough. In fact, after the detection, it was confirmed that HCL gas remained unreacted in the gas flowing out of the outlet-side pipe 17.

As described above, according to the above-described Examples of the present invention, it is possible to provide a detecting device for a hydrogen halide gas and an absorbing apparatus for a hydrogen halide gas each with a simple structure.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A detecting device for a hydrogen halide gas, comprising:
an insulating support;
a detecting member supported on the insulating support and containing an absorbent which reacts with the hydrogen halide gas to produce water; and
a pair of electrodes attached respectively to both ends of the detecting member and configured to measure a change in an electric resistance value or an electrostatic capacitance of the detecting member, caused by the production of water due to a reaction between the hydrogen halide gas and the absorbent in the detecting member.

2. The detecting device according to claim 1, wherein the absorbent comprises at least one material selected from the group consisting of lithium composite oxides and hydroxides of alkaline earth metals.

3. The detecting device according to claim 1, wherein the lithium composite oxide is a lithium silicate expressed by chemical formula, Li₄SiO₄.

4. The detecting device according to claim 1, wherein the detecting member is a ribbon member of an aggregate of a plurality of granular absorbents each made of at least one material selected from the group consisting of lithium composite oxides and hydroxides of alkaline earth metals, and is brought into contact with the pair of electrodes at vicinities of both ends of the ribbon member.

5. The detecting device according to claim 47 wherein the granular absorbents have an average diameter of 1 μm to 3 mm.

6. The detecting device according to claim 4, wherein the ribbon member is further surrounded by an insulating member fixed onto the insulating support so as to stride over the pair of electrodes.

7. An absorbing apparatus for a hydrogen halide gas, comprising:
a cylindrical absorbing column made of an insulating material, through which a gas to be treated containing a hydrogen halide gas is allowed to flow, and having an inlet and an outlet of the gas to be treated;
a plurality of absorbents filled in the absorbing column and which reacts with the hydrogen halide gas to produce water; and
a pair of electrodes provided at a section of the absorbent situated on at least the outlet side of the absorbing column, and configured to measure a change in an electric resistance value or an electrostatic capacitance of the absorbents, caused by the production of water due to a reaction between the hydrogen halide gas and the absorbents.

8. The absorbing apparatus according to claim 7, wherein the absorbents comprise at least one material selected from the group consisting of lithium composite oxides and hydroxides of alkaline earth metals.

9. The absorbing apparatus according to claim 8, wherein the lithium composite oxide is a lithium silicate expressed by chemical formula, Li₄SiO₄.

10. The absorbing apparatus according to claim 8, wherein the absorbents are granules containing at least one material selected from the group consisting of lithium composite oxides and hydroxides of alkaline earth metals, and a binder.

11. The absorbing apparatus according to claim 10, wherein the granules have an average diameter of 50 μm to 30 mm.

12. The absorbing apparatus according to claim 10, wherein the binder is at least one selected from the group consisting of polyvinyl alcohol, polyvinyl butyral, wax, paraffin and carboxymethylcellulose.

13. The absorbing apparatus according to claim 10, wherein the binder is contained in the absorbents at a ratio of 0.1 to 20% by weight.
